(19) United States
(12) Patent Application Publication
(10) Pub. No.: US 2012/0220844 A1
(43) Pub. Date: Aug. 30, 2012

(54) Regional Saturation Using Photoacoustic Technique

(75) Inventor: Clark R. Baker, JR., Newman, CA (US)

(73) Assignee: Nellcor Puritan Bennett LLC, Boulder, CO (US)

(21) Appl. No.: 13/036,503

(22) Filed: Feb. 28, 2011

(51) Int. Cl. A61B 5/1455

(52) U.S. Cl. 600/328; 600/323; 600/340

(57) ABSTRACT

Methods and systems are provided for determining the oxygen saturation of a region in a patient's body using photoacoustic spectroscopy techniques. One embodiment includes determining an interrogation region, or a region in a patient to be monitored, and using a photoacoustic sensor to emit modulated light in the interrogation region. The modulated light may be absorbed by different absorbers, such as oxygenated hemoglobin and deoxygenated hemoglobin, in the interrogation region. The absorbed light results in an acoustic response which is detected by the photoacoustic sensor. Based on a non-pulsatile component of the acoustic response, the regional oxygen saturation at the interrogation region is calculated.

### CROSS REFERENCE TO RELATED APPLICATIONS

This application is related to U.S. Patent Application No. 2006/0253016, which was filed on Nov. 18, 2005, and is hereby incorporated by reference for all purposes.

### BACKGROUND

The present disclosure relates generally to medical devices and, more particularly, to measuring regional saturation using photoacoustic spectroscopy techniques.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

For example, clinicians may wish to monitor a patient's blood oxygen saturation in a particular region of the patient's body. Such a measurement, referred to as regional saturation, is commonly used to monitor the oxygen saturation in a patient's brain when the patient is under anesthesia and/or undergoing a cardiopulmonary surgical procedure. Normal or expected values of regional saturation in the brain may indicate that the patient is maintaining appropriate cerebral hemispheric blood oxygen saturation levels during surgery. Deviation from normal values may alert a clinician to the presence of a particular clinical condition. For instance, oxygen desaturation in a patient's brain may indicate that the patient's brain is not sufficiently receiving hemoglobin. Such an indication may enable a health care provider to take the necessary actions to prevent hypoxia in the brain which might result in brain dysfunction and damage.

Regional saturation may be determined using a cerebral oximeter, which involves using a non-invasive sensor that passes light through a portion of the patient's tissue and photo-electrically senses the absorption and scattering of light in the tissue. The amount of light that is absorbed and/or scattered is used to estimate the amount of blood constituent in the tissue. The pulsatile component of the oximeter signal may be indicative of an arterial oxygen perfusion (i.e., an absolute blood oxygen saturation level of the whole body), and the non-pulsatile component of the signal may be indicative of a regional or local perfusion (i.e., regional saturation of the interrogated region in the body).

However, using techniques such as pulse oximetry to determine regional saturation may sometimes be imprecise due to the scattering characteristics of light as it is impinged in tissue. The uncertainty of light scattering in tissue may limit the calibration of pulse oximeters when measuring regional saturation, resulting in a lack of specificity in the interrogated region. For instance, in some pulse oximeter systems, specificity is limited to fairly large regions of tissue (e.g., half the patient's forehead). Such a lack of specificity may limit the accuracy and/or information provided using the cerebral oximetry technique.

### BRIEF DESCRIPTION OF THE DRAWINGS

Advantages of the disclosed techniques may become apparent upon reading the following detailed description and upon reference to the drawings in which:

FIG. 1 illustrates a pulse oximetry device measuring a patient's cerebral oxygen saturation;

FIG. 2 illustrates a simplified block diagram of photoacoustic spectroscopy sensor, according to an embodiment; and

FIG. 3 illustrates a flow chart depicting a process using photoacoustic spectroscopy to determine regional saturation in a patient.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to monitoring regional saturation in a patient using photoacoustic spectroscopy. Regional saturation may involve non-invasively estimating the oxygenation of an interrogated region of a patient. Blood oxygenation is generally taken by a pulse oximetry device such as a finger-sensor pulse oximeter and may provide the arterial blood oxygenation level of the body as a whole. However, the arterial blood oxygenation level of a patient may not provide sufficient detail regarding the oxygenation levels of particular regions of the patient's body.

Certain surgical procedures (e.g., cardiopulmonary, neurological, or vascular surgeries) may be performed in low blood flow or low blood pressure conditions, and may also involve blood loss. Moreover, during such surgical procedures, the patient is typically anesthetized, further complicating the detection of blood loss. Such conditions may all contribute to potential decreases of oxygen delivery to the brain. As the brain is relatively intolerant to oxygen deprivation, insufficient oxygenation to the brain may result in hypoxia in the brain. Health practitioners may monitor the oxygen saturation of the brain to determine if and when oxygen saturation levels in the patient's brain fall beneath a certain threshold.

Systemic measurements, or measurements representing a patient's whole body, may be insufficient for accurately determining regional saturation of a patient. Such systemic measurements may include blood pressure, urine output, or general pulse oximetry, for example. A general pulse oximetry measurement may refer to an estimation of the pulsatile component of blood flow through the interrogated region (e.g., typically, appendages such as a finger or ear lobe), which corresponds to the arterial saturation in the patient's whole body. However, the arterial saturation may be insufficient in indicating the oxygen saturation at a particular region of a patient's body. For instance, while the arterial saturation of a patient's body may indicate that the patient's body has a normal oxygen saturation level, particular regions of the patient, such as the patient's brain, may not have sufficient oxygenation. Therefore, site-specific issues may still occur, even when the patient's arterial saturation measurement is within a normal range.

Moreover, oximetry techniques which are configured to measure oxygen saturation at a patient's brain have limited specificity due to the unpredictable scattering characteristics of light as it is impinged in tissue. For example, as light is emitted towards patient tissue, it may be scattered in various directions by the tissue and/or blood. Not all of the scattered light may be detected at the photodetector, possibly resulting in determining an inaccurate ratio of absorbed and scattered light. The uncertainty of light scattering in tissue may limit the calibration of oximeters when measuring cerebral saturation, resulting in a lack of specificity in the interrogated region. For instance, in some oximeter systems, specificity is limited to fairly large regions of tissue, such as an entire hemisphere of the patient's cerebrum.

One or more embodiments of the present techniques include using photoacoustic spectroscopy to measure the cerebral oxygen saturation of a patient. Photoacoustic spectroscopy involves emitting modulated light into a tissue such that the emitted light is absorbed by certain components of the tissue and/or blood. The light modulation pattern may comprise short discrete bursts of light with durations generally under one microsecond, or continuous frequencies generally above 1 MHz, such as chirp signals. Tissue and/or blood in an interrogated region may absorb the emitted light and generate kinetic energy, which results in pressure fluctuations at the interrogated region. The pressure fluctuations may be detected in the form of acoustic radiation (e.g., ultrasound) by a sensor (e.g., a photoacoustic transducer). As different absorbers and concentrations of absorbers at an interrogated region may have different absorption properties, the amplitude of the detected acoustic radiation may be correlated to a density or concentration of a particular absorber.

Using photoacoustic spectroscopy to determine the oxygen saturation of an interrogated region may provide certain advantages over the conventional oximetry techniques. For example, photoacoustic spectroscopy may provide more information corresponding to the depth (e.g., a depth in the tissue relative to the photoacoustic spectroscopy sensor; z-direction) of a detected acoustic radiation. More specifically, a phase difference or time delay between the detected acoustic radiation and the emitted light modulation may indicate a depth in the interrogated region. In some embodiments, more than one photoacoustic sensor may be used to focus in multiple lateral axes of an interrogated region (e.g., in-plane with the tissue surface and/or the sensor; x- and y-directions) which may be combined to provide three-dimensional images of an interrogated region.

Furthermore, photoacoustic spectroscopy techniques may provide increased accuracy in determining the oxygen saturation of an interrogated region, as photoacoustic techniques are based on the absorption of light by tissue and/or blood, rather than on the scattering of light, which may result in less specificity as previously discussed. For instance, while the spatial resolution using conventional oximetry techniques may be limited to a few millimeters in regions of diffuse vasculature clue to the uncertainty of light scattering, determining regional saturation using photoacoustic spectroscopy may have a spatial resolution of 1 mm or less, particularly when interrogating larger vessels which more strongly absorb light.

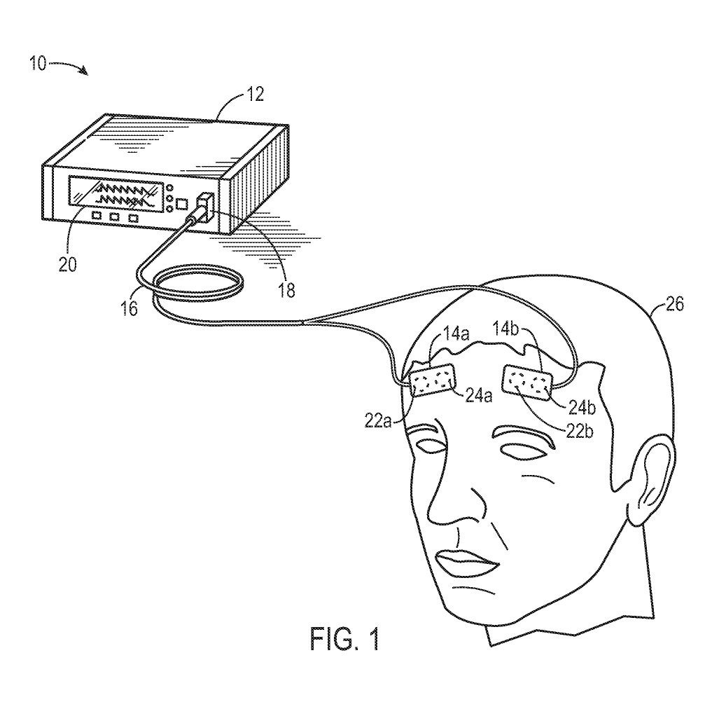

FIG. 1 illustrates one example of a regional saturation system 10 suitable for using photoacoustic spectroscopy techniques to measure the oxygen saturation of an interrogated region in a patient 26. The photoacoustic spectroscopy system 10 may include a patient monitor 12 and a photoacoustic spectroscopy sensor 14. Some regional saturation systems 10 may include more than one photoacoustic spectroscopy sensor 14a and 14b, as illustrated in FIG. 1, to interrogate different regions (e.g., left and right hemispheres, different portions of one hemisphere, etc.) of the patient's cerebral tissue 26. Additionally, in some embodiments, using more than one sensor may provide data for generating three-dimensional images of an interrogated region. A sensor cable 16 may connect the patient monitor 12 to the sensor 14, and may include two or more cables. One of the cables within the sensor cable 16 may transmit an input signal from the patient monitor 12 to emit modulated light into the patient cerebral tissue 26 by an emitter 22 on the sensor 14. The input modulated light may propagate through the cerebral tissue 26 and may be translated into kinetic energy, resulting in an acoustic response. The acoustic response may be received as an output signal by a detector 24 on the sensor 14.

The detector 24 may transmit a signal indicative of the detected acoustic response to the patient monitor 12, where the cerebral oxygen saturation may be calculated. For instance, the signal may be transmitted to the patient monitor 12 by the cable 16 which couples to the monitor 12 via a connection 18. Based on signals received from the sensor 14, the patient monitor 12 may determine a regional oxygen saturation of the interrogated region to be displayed on a display 20.

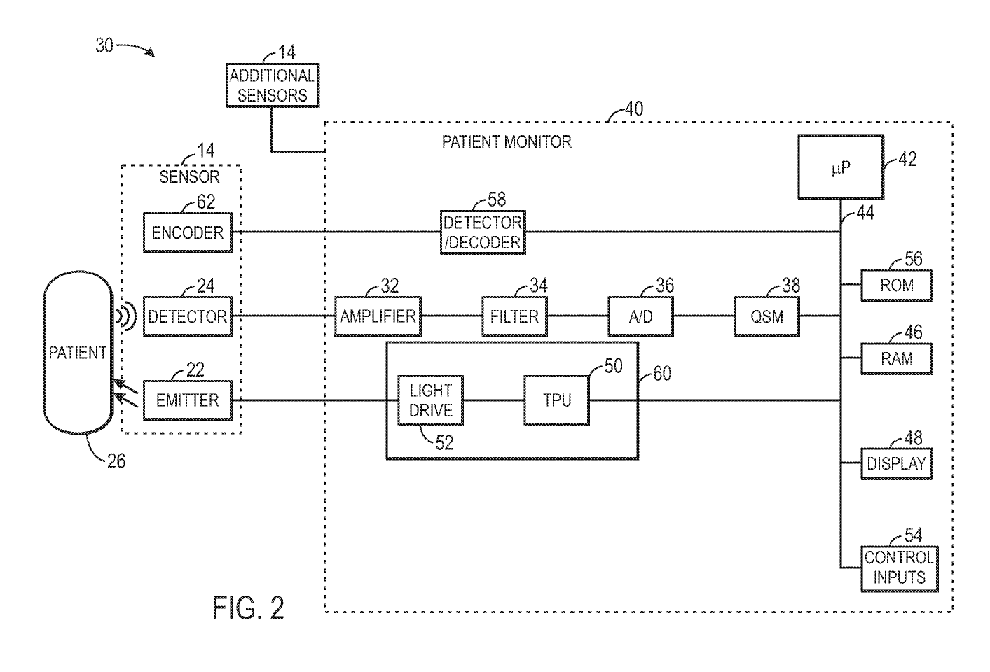

FIG. 2 illustrates a simplified block diagram of the regional saturation system 10 illustrated in FIG. 1. As discussed, the regional saturation system 10 may use photoacoustic techniques to monitor the oxygen saturation level of a patient 26 in a particular region of the patient 26. A region of a patient 26, also referred to as an interrogated region, may refer to any region of interest in the body of the patient 26. For example, an interrogated region may include the brain, a hemisphere of the brain, a particular location in the brain, bodily organs such as the abdomen, kidney, liver, any particular locations in bodily organs, or compartments in the body of the patient 26, such as the abdominal compartment or the chest, in which a medical practitioner monitors a venous oxygen saturation level. An oxygen saturation measurement of a region (referred to as regional saturation) may be differentiated from a whole-body measurement (e.g., such as from a pulse oximetry finger sensor measurement) in that the regional saturation is an estimation of the venous oxygen saturation in a particular region of interest in the body of the patient 26, while a conventional oximetry measurement is an estimation of the arterial oxygen saturation in the whole body. Moreover, a region may be a relatively small and/or defined volume within the body of the patient 26, and may be measured by one photoacoustic sensor 14. Additionally, in some embodiments, more than one sensor 14 may be used to measure a region, depending on the size of the region of interest and/or the configuration of the sensor 14. For instance, region of interest may have a larger volume than a volume typically monitored by a single sensor 14, and multiple sensors 14 may be used to interrogate the entire region of interest.

In some embodiments, using photoacoustic techniques to monitor regional saturation may be employed while the patient 26 is undergoing a surgical procedure. The present techniques of using photoacoustic spectroscopy to determine regional oxygen saturation can be applied to any region of a patient 26. While the patient's cerebrum tissue is discussed as one example of an interrogated region, the present techniques are not limited to patient cerebrum tissue. For example, as previously discussed, in some embodiments, the regional saturation system 10 may be used to determine the oxygen saturation in different organs of the patient 26.

The system 10 includes a photoacoustic spectroscopy sensor 14 with a light emitter 22 and an acoustic detector 24. The sensor 14 may emit a light in a continuous manner or in a pulsed manner, depending on the configuration of the system 10, the region of the patient 26 to be interrogated, and/or the length of time for regional interrogation. The emitter 22 may include one or more light emitting diodes (LEDs) adapted to transmit one or more wavelengths of light, and the detector 24 may include one or more ultrasound transducers configured to receive ultrasound waves generated by the tissue in response to the emitted light and to generate a corresponding electrical or optical signal. In specific embodiments, the emitter 22 may be a laser diode or a vertical cavity surface emitting laser (VCSEL). The laser diode may be a tunable laser, such that a single diode may be tuned to various wavelengths corresponding to a number of absorbers. Depending on the particular arrangement of the photoacoustic sensor 14, the emitter 22 may be associated with an optical fiber for transmitting the emitted light into the tissue.

The light emitted by the emitter 22 may be emitted at suitable wavelengths based on the absorption coefficients of certain constituents in the blood, the interrogation region of the patient 26, and/or the distance of the interrogated region from the sensor 14. For example, in some embodiments, the light may be emitted at wavelengths which are differently absorbed by the different blood constituents in the interrogated region (e.g., oxygenated hemoglobin and deoxygenated hemoglobin), and insignificantly absorbed by certain irrelevant components in the interrogated region (e.g., water). In some embodiments, the different wavelengths of light may be multiplexed or emitted sequentially from the emitter 22, such that the resulting acoustic responses include spatial variations corresponding to each emitted wavelength. Ratios of acoustic response variations along the axial or lateral dimensions may then be used to determine the ratio of absorption coefficients in the interrogated region. If light is modulated to wavelengths which significantly absorb hemoglobin, the ratio may indicate the oxygen saturation of the interrogated region.

Furthermore, in some embodiments, light may be emitted at suitable wavelengths to interrogate regions having different depths within the tissue (e.g., different distances from the sensor 14). In some embodiments, wavelengths of light between about 600 nm to about 950 nm may be suitable for cerebral oxygen saturation monitoring, as the 600 nm to 950 nm wavelength range may penetrate through a patient's skull and may be absorbed differently by oxyhemoglobin and deoxyhemoglobin in the cerebrum tissue once it has penetrated the skull. For example, an emitter 22 may emit light modulated to a wavelength of approximately 800 nm to be absorbed by oxyhemoglobin and light modulated to a wavelength of approximately 700 to be absorbed by deoxyhemoglobin. Furthermore, wavelengths below about 950 nm are generally not significantly absorbed by water. Wavelengths between about 450 nm to about 600 nm may be more significantly absorbed by hemoglobin and may be used for monitoring regional saturation of more superficial tissues, or at an interrogated region with a comparatively smaller distance from the sensor 14.

In some embodiments, the light source at the emitter 22 may also be modulated with a modulation pattern suitable for interrogating different regions of the patient 26. For example, the axial resolution of photoacoustic system may be of proportional to the ultrasound wavelength in tissue. Given an ultrasound velocity of approximately 1500 m/see in tissue, a 1.5 Mhz modulation frequency may be have a wavelength in tissue of 1 mm. Higher modulation frequencies or shorter discrete bursts may be suitable for interrogating tissue regions at shorter depths and distances from the sensor 14, while lower frequencies or longer discrete bursts may result in less ultrasound absorption by tissue and may be more suitable for interrogating deeper tissue regions where higher signal levels are worth the reduced resolution.

The acoustic detector 24 may include an acoustic transducer or another receiver suitable for receiving an acoustic response generated by the tissue when exposed to the emitted light. In some embodiments, the detector 24 may also be suitable to receive other types of responses such as a pressure fluctuation, a thermal response, or any other non-optical response generated by the conversion of absorbed light energy into kinetic energy. An acoustic response will be used herein as one example of the tissue's response to the emitted light, which is detected by the detector 24. In some embodiments, the detector 24 may output a voltage signal proportional to the acoustic response generated in the tissue. This output voltage signal may be a DC, non-pulsatile component of the acoustic response received at the detector 24. The detector 24 may use, for example, a frequency mixer, to lock onto the frequency of the acoustic wave and convert the phase and amplitude of the acoustic wave to a voltage signal.

In one embodiment, the detector 24 may be a low finesse Fabry-Perot interferometer, which may include a thin polymer sensing film mounted at the tip of an optical fiber. The thin sensing film may allow a higher sensitivity to be achieved than a thicker sensing film. Using a Fabry-Perot polymer film interferometer, an incident acoustic wave emanating from the probed tissue may modulate the thickness of the thin polymer film and the phase difference of the light reflected from the two sides of the polymer film. The light reflection produces a corresponding intensity modulation of the light reflected from the film. Accordingly, the acoustic wave may be converted to optical information, which may be transmitted through an optical fiber to a suitable optical detector. The change in phase of the detected light may be detected via an appropriate interferometry device.

In some embodiments, the photoacoustic spectroscopy sensor 14 may be configured to be moved during operation of the regional saturation system 10. For instance, to measure larger regions than a region interrogated by a single sensor 14, the sensor 14 may be manually or automatically moved over a larger region to be interrogated. In some embodiments, the system 10 may use object recognition or image processing software to detect the location of the sensor 14, a position, orientation, and/or elevation of the tissue site, etc. For example, such techniques are discussed in U.S. Patent Application No. 2006/0253016, which is hereby incorporated by reference for all purposes. Furthermore, in some embodiments, a regional saturation system 10 may include more than one photoacoustic spectroscopy sensor 14. Each of the multiple sensors 14 in the system 10 may include an emitter 22 and detector 24 and may be suitable for interrogating different regions of a patient 26. To measure oxygen saturation at relatively larger regions, more sensors 14 may be used concurrently, and the information obtained from the multiple sensors 14 may be combined to determine an oxygen saturation of an entire region of interest. For example, in some embodiments, a system 10 may have four different sensors 14, and each of the four sensors 14 may be used to interrogate four different regions of a patient's brain, such that a larger combined region of the patient's brain is interrogated. Moreover, in systems 10 having more than one sensor 14, each of the sensors 14 may be suitable for concurrently emitting different wavelengths of light during an operation of the system 10.

The regional saturation system 10 may also include a monitor 40 which may receive signals from the photoacoustic spectroscopy sensor 14. The monitor 40 may determine the oxygen saturation of an interrogated region based on the signals received by the photoacoustic spectroscopy sensor 14. The monitor 40 may include a microprocessor 42 coupled to an internal bus 44. Also connected to the bus 44 may be a RAM memory 46 and a display 48. A time processing unit (TPU) 50 may provide timing control signals to light drive circuitry 52, which controls the emission of light by the sensor 14 when activated, and, if multiple sensors 14 and/or light sources are used, the multiplexed timing for the different light sources. TPU 50 may also control the gating-in of signals from the sensor 14. These signals are sampled at the proper time, depending at least in part upon which light sources are activated, if multiple sensors 14 are used, and/or which wavelengths of light are being emitted if the emitted light is modulated at more than one wavelength. In some embodiments, the signal received from the sensor 14 may be passed through one or more signal processing elements, such as an amplifier 32, a low pass filter 34, and/or an analog-to-digital converter 36. Furthermore, in some embodiments, digital data may be stored in a suitable storage component in the monitor 40, for example, in the queued serial module (QSM) 38, RAM 46, or ROM 56.

The TPU 40 and the light drive circuitry 42 may be part of a modulator 60, which may modulate the drive signals from the light drive circuitry 52 that activate the LEDs or other emitting structures of the emitter 22. The modulator 60 may be hardware-based, software-based, or some combination thereof. For example, a software aspect of the modulator 60 may be stored on the memory 46 and may be executed by the processor 42. In some embodiments, the modulator 60 may be configured to modulate a continuous wave light emitted from the photoacoustic spectroscopy sensor 12, and may be any modulator suitable for modulating a continuous wave source at a low power. In some embodiments, the modulator 60 may be suitable for modulating higher power pulses of light. While the modulator 60 is depicted as in the monitor 40, in some embodiments, the modulation function may be performed by a modulator disposed in the photoacoustic spectroscopy sensor 14. In one embodiment, the modulation and detection features may both be located within the sensor 14 to reduce the distance traveled by the signals, and to reduce potential interferences. In some such embodiments, the sensor cable 16 may be replaced by a wireless communication link.

In an embodiment, based at least in part upon the signals generated by the detector 24 in response to the detected acoustic waves, the microprocessor 42 may calculate the oxygen saturation of an interrogated region using various algorithms. In some embodiments, the microprocessor 42 calculates regional saturation based on the DC, non-pulsatile component of the detected acoustic response. For example, in some embodiments, the microprocessor 42 may receive the voltage signal output by the detector 24. Furthermore, patient conditions may be also analyzed based on control inputs 54 input by a user. For instance, a caregiver may input a patient's clinical condition, interrogated region, surgical procedure, or any other information which may be relevant in analyzing the oxygen saturation of an interrogated region.

The algorithms used to calculate regional saturation may employ certain coefficients, which may be empirically determined, and may correspond to the wavelength of light used. In addition, the algorithms may employ additional correction coefficients. In one embodiment, an acoustic response generated in response to interrogation by a sensor 14 is represented in accordance with the equation:

$${E}_{m} = {\sum }_{t} {A}_{m} {P}_{t - {d}_{m} / c} {P}_{t - Δ t} \qquad ( 1 )$$

where Eₘ is proportional to the cross-correlation of the emitted light modulation pattern, P_{b} generated by the modulator 60 at time-delay Δt with the detected modulation pattern P_{t-dm/c}, Aₘ is the amplitude of the light absorbed by a segment (e.g., a depth in an interrogated region) in, dₘ is the distance (e.g. depth) from the segment m to the detector, and c is the ultrasound velocity in tissue. In accordance with such an equation, the magnitude of Eₘ may be maximized by setting Δt equal to dₘ/c. This process may then be repeated for each segment of the interrogated region. The value of Eₘ at each time-delay Δt may then be stored. In some embodiments, the time-delay for each segment may be used to determine the depths of the segments in the interrogation region with respect to the location of the sensor 14. Further, in some embodiments, information from different segments (e.g., different depths in an interrogation region) may be used to construct a three-dimensional image of the interrogation region.

In some embodiments, the monitor 40 (e.g., the microprocessor 42) may apply various algorithms to the voltage signals output by the detector 24 to quantitatively calculate the oxygen saturation of an interrogated region. For instance, an interrogation region may first be irradiated by light from the emitter 22 having an optical wavelengths λ₁ corresponding to a constituent of interest C₁ followed by a second irradiation of the light from the emitter 22 at an optical wavelength λ₂ corresponding to a second constituent of interest C₂. The two acoustical responses corresponding to the light waves λ₁ and λ₂ may then be detected by the detector 24 and processed. The signals output by the detector 24 may first be normalized by using an average signal corresponding to, for example, a theoretical tissue devoid of embedded objects, in order to compensate for differences in light fluence between the two wavelengths in the interrogation region. Absorption coefficients for the constituents of interest in the interrogated region may then be used as follows:

$$\frac{[ {C}_{1} ]}{[ {C}_{1} ] + [ {C}_{2} ]} = \frac{{μ}_{a} ( {λ}_{2} ) {ɛ}_{{C}_{2}} ( {λ}_{1} ) - {μ}_{a} ( {λ}_{1} ) {ɛ}_{{C}_{2}} ( {λ}_{2} )}{{μ}_{a} ( {λ}_{1} ) Δɛ ( {λ}_{2} ) - {μ}_{a} ( {λ}_{2} ) Δɛ ( {λ}_{1} )} \qquad ( 2 )$$

where [C₁] is the approximate relative concentration of the first constituent of interest (e.g., oxyhemoglobin) in a carrier medium such as blood, [C₂] is the approximate relative concentration of the second constituent of interest (e.g., deoxyhemoglobin) in the carrier medium, ε_{c}_{₁} is the absorption coefficient of the first constituent of interest, ε_{c}_{₂} is the absorption coefficient of the second constituent of interest, μₐ is the measured relative absorption at each wavelength, and Δε=ε_{c}_{₁}−ε_{c}_{₂}. For example, the ratio of oxyhemoglobin to total hemoglobin (deoxy-plus oxyhemoglobin) may be found by utilizing their known or measured absorption coefficients in equation (2). By varying the wavelengths used for observation, various different types of constituent measurements may be derived based on the observation that different types of constituents absorb light at different wavelengths.

Such algorithms and coefficients relating to the above equations may be stored in a ROM 56 or other suitable computer-readable storage medium and accessed and operated according to microprocessor 42 instructions. In addition, the sensor 14 may include certain data storage elements, such as an encoder 62, that may encode information related to the characteristics of the sensor 14, including information about the emitter 22 and/or the detector 24. The information may be accessed by detector/decoder 58, located on the monitor 40. Furthermore, if multiple sensors 14 are in use and/or multiple regions are being monitored, the microprocessor 42 may be suitable for concurrently processing the voltage signals output by the detector 24 to calculate the regional saturation of the interrogated region.

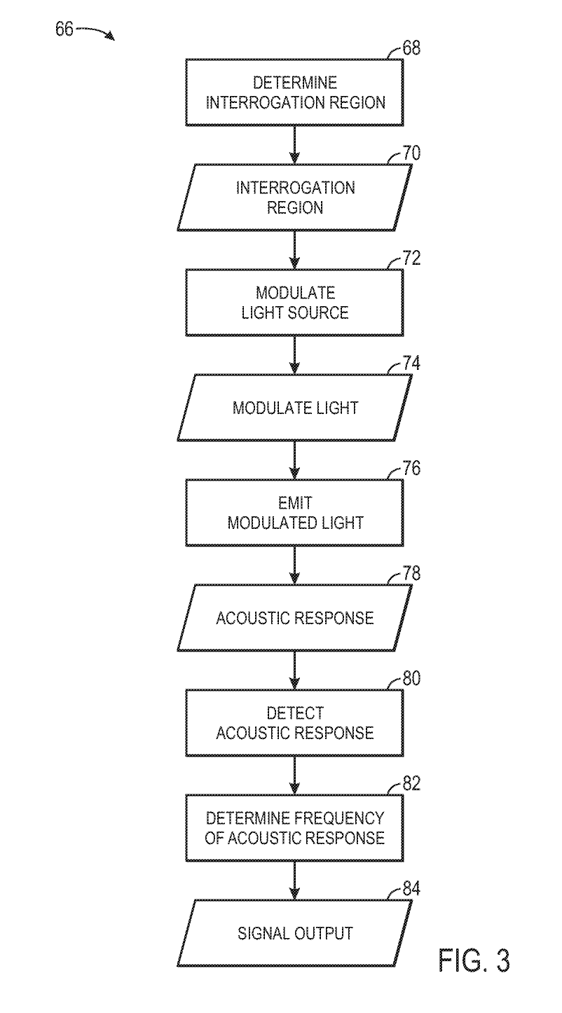

A method for using photoacoustic spectroscopy to determine regional oxygen saturation in a patient is provided in FIG. 3. The process 66 may begin by determining (block 68) a region in the patient 26 to be interrogated. The interrogation region 70 may be any region of interest (e.g., brain, organ, superficial tissue, and/or portions of the brain or other organs or superficial tissue) of the patient's body in which regional oxygen saturation is measured. The process 66 may include modulating (block 72) a light source of a sensor 14 (as in FIG. 2) to emit a modulated beam 74 at a suitable wavelength. The modulation process may involve controlling the operating current of, for example, a laser diode in the emitter 22, which may be substantially controlled by light drive circuitry 52 in a modulator 60 of a regional saturation system 10. The modulation frequency may be based on various factors, including the physiology, size, and/or other condition of the interrogation region 70, the physiological condition of the patient 26, the absorbers of interest at the interrogation region 70, and/or the photoacoustic spectroscopy system limitations. For example, if a region in the patient's cerebrum is to be interrogated, the emitted light may be at wavelengths (e.g., between about 600 nm to about 950 nm), which may penetrate through the patient's skull and absorbed by hemoglobin in the cerebrum tissue, and the modulation pattern may be determined based on tradeoffs between axial resolution and signal levels,

Once the sensor 14 emits (block 76) the modulated light towards the interrogation region 70, the light energy may be absorbed by certain components of the tissue and/or blood (e.g., absorbers) based on the concentration of absorbers at the interrogation region 70 and the absorption coefficients of the absorbers. The absorbed light energy may be converted to kinetic energy, which generates an acoustic response 78 (e.g., an acoustic wave) in the tissue at the interrogation region 70. The acoustic response 78 may be received (block 80) by a detector 24 in the photoacoustic spectroscopy sensor 14. In some embodiments, the detector 24 may determine the frequency (block 82) of the detected acoustic response 78. The detector 24 may determine the frequency of the acoustic response 78 based on a frequency of the modulated light 74. Thus, the detector 24 may perform a delay-sensitive detection process by determining the amplitude of the acoustic response 78, as well as the time-delay between the acoustic response 78 and the waveform of the emitted modulated light 74.

Based on a comparison of the waveform of the emitted modulated light 74 and the detected acoustic response 78, the amplitude component of the acoustic response 78 and the phase shift or time delay between the acoustic response 78 and the emitted modulated light 74 may provide information as to the concentration and/or location of the absorbers being measured. The amplitude component of the acoustic response 78 may provide information corresponding to the concentration of absorbers being measured, as the intensity of the acoustic reaction 78 may be proportional to the amount of light absorbed by the absorbers having a certain absorption coefficient. The phase component of the acoustic response 78 may provide information corresponding to the location of the absorbers being measured. More specifically, the phase component may be a time delay between the modulated light 74 and the acoustic response 78. The monitor 40 may determine (e.g., based on algorithms executed by a microprocessor 42, such as that provided in equation (1)), that the sensor 14 is measuring absorbers at a certain depth in the tissue based on the phase information of the acoustic reaction 78. The detector 24 may output a voltage signal 84 of the amplitude and phase information of the acoustic response 78. This voltage signal 84 may represent the DC, non-pulsatile component of the acoustic reaction 78 to the emitted light 74. As discussed, this signal 84 may be used by the monitor 40 for further processing and/or analyses of regional saturation. For example, the monitor 40 may calculate, using various algorithms, such as in equation (2), the concentration of oxyhemoglobin and deoxyhemoglobin in an interrogated region.

What is claimed is:

1. A method, comprising:
modulating a light source in a photoacoustic spectroscopy sensor to emit a light having a first wavelength absorbable by a first absorber in the interrogation region;
emitting the modulated light towards an interrogation region in a patient;
detecting from the interrogation region an acoustic response to the emitted modulated light; and
determining a regional oxygen saturation of the interrogation region based on a non-pulsatile component of the acoustic response.

2. The method of claim 1, wherein the interrogation region comprises a region in a patient's brain.

3. The method of claim 1, comprising selecting multiple interrogation regions to monitor regional oxygen saturation in a combined region that is spatially larger than one interrogation region.

4. The method of claim 1, comprising modulating a plurality of light sources, each of the plurality of light sources in one of a plurality of photoacoustic spectroscopy sensors, to emit a plurality of lights, each having a wavelength absorbable by one or more absorbers in the interrogation region.

5. The method of claim 1, wherein modulating the light source is based on one or more of the selected interrogation region, a clinical condition of the patient, and a length of time in which regional oxygen saturation is to be monitored.

6. The method of claim 1, wherein modulating the light source comprises modulating the light to the first wavelength and a second wavelength, wherein the first wavelength is significantly absorbable by oxygenated hemoglobin in the interrogation region and wherein the second wavelength is significantly absorbable by deoxygenated hemoglobin in the interrogation region.

7. The method of claim 6, comprising multiplexing the light modulated to the first wavelength and the light modulated to the second wavelength.

8. The method of claim 1, wherein modulating the light source comprises modulating a continuous light source.

9. The method of claim 1, wherein modulating the light source comprises modulating a pulsed light source.

10. The method of claim 1, wherein the non-pulsatile component of the acoustic response comprises a frequency component of the acoustic response.

11. The method of claim 1, comprising focusing the detected acoustic response on each of a plurality of depths in the interrogation region to produce a plurality of interrogated depths.

12. The method of claim 11, comprising forming a three dimensional image of the interrogation region from the plurality of interrogated depths by also focusing the detected acoustic response on each of a plurality of lateral positions.

13. A regional saturation system, comprising:
one or more photoacoustic spectroscopy sensors, wherein each of the one or more photoacoustic spectroscopy sensors comprises:
a light source configured to be modulated to emit one or more wavelengths of light into an interrogation region of a patient; and
a detector configured to receive a response wave generated in the interrogation region in response to the light emitted by the light source, wherein the response wave is non-optical; and
a processor configured to determine a regional concentration of an absorber in the interrogation region based on the response wave and based on the one or more wavelengths of light emitted into the interrogation region.

14. The regional saturation system of claim 13, wherein the light source is configured to be modulated to emit light comprising a wavelength between approximately 450 nm to approximately 950 nm.

15. The regional saturation system of claim 13, wherein the response wave is one or more of a pressure wave, an acoustic wave, or a thermal wave.

16. The regional saturation system of claim 13, wherein the detector is configured to determine a frequency of the response wave based on the light emitted into the patient's tissue.

17. The regional saturation system of claim 16, wherein the detector is configured to output a voltage signal comprising one or more of frequency information, amplitude information, and phase information of the response wave.

18. The regional saturation system of claim 13, comprising memory storing algorithms directed to calculating the concentration of the absorber and the depth of the absorber, wherein the processor is capable of accessing the memory to execute the algorithms.

19. The regional saturation system of claim 13, comprising a display configured to display the regional concentration of the absorber in the interrogation region.

20. A regional saturation patient monitor, comprising:
a modulator configured to modulate a light source;
data processing circuitry configured to receive a response to an emission of the light source and determine a non-pulsatile component of the response, wherein the response comprises non-optical data; and
a processor configured to utilize the non-pulsatile component to calculate a regional oxygen saturation of a patient at an interrogation region.

21. The regional saturation patient monitor of claim 20, comprising a user input configured to input one or more of a modulation parameter of the light source, a condition of the patient, and a location of the interrogation region.

22. The regional saturation patient monitor of claim 20, wherein the non-optical data comprises one or more of a pressure wave, an acoustic wave, or a thermal wave.
